(19) United States
(12) Patent Application Publication
(10) Pub. No.: US 2026/0076331 A1
(43) Pub. Date: Mar. 19, 2026

(54) PEANUT CULTIVAR 'IPG 110'

(71) Applicant: International Peanut Group LLC, Brownfield, TX (US)

(72) Inventors: Jimmy D. Gregory, Brownfield, TX (US); Dylan Quincy Wann, Brownfield, TX (US)

(21) Appl. No.: 19/330,659

(22) Filed: Sep. 16, 2025

Related U.S. Application Data
(60) Provisional application No. 63/695,253, filed on Sep. 16, 2024.

(51) Int. Cl. A01H 6/54; A01H 5/10

(52) U.S. Cl. CPC A01H 6/541; A01H 5/10

(57) ABSTRACT

A peanut cultivar designated IPG 110 is disclosed herein. The present invention provides seeds, plants, and plant parts derived from peanut cultivar IPG 110. Further, it provides methods for producing a peanut plant by crossing IPG 110 with itself or another peanut variety. The invention also encompasses any peanut seeds, plants, and plant parts produced by the methods disclosed herein, including those in which additional traits have been transferred into IPG 110 through genetic engineering, gene editing, mutagenesis, or by breeding IPG 110 with another peanut cultivar.

### CROSS-REFERENCE TO RELATED APPLICATIONS

This application claims priority to U.S. Provisional Application No. 63/695,253 filed on Sep. 16, 2024, the contents of which are incorporated by reference in its entirety.

### BACKGROUND OF THE INVENTION

The present invention relates to a new and distinctive peanut (Arachis hypogaea L.) cultivar, designated IPG 110.

The peanut is an annual herbaceous plant of the legume family. Originally cultivated in South America and the eastern slopes of the Andes mountains, peanut is now grown worldwide in the tropic and temperate zones and is recognized as one of the major oilseed crops and as a rich source of protein. In the United States, peanut production is concentrated primarily in the Southeast (Alabama, Florida, Georgia, Mississippi, South Carolina), the Southwest (New Mexico, Oklahoma, Texas), and Virginia and North Carolina.

The peanut plant grows best in light, sandy soil and requires four to five months of warm weather and an annual rainfall of 20 to 39 inches, or the equivalent in irrigation water. The pea-like yellow flowers form in axillary clusters and only bloom for a short time. Following self-pollination, the stalk at the base of the ovary, called the pedicel, elongates rapidly and turns downward to bury the fruits one to several inches below the ground surface. The peanut pods complete their development 120 to 160 days after planting. During harvest, the entire plant including the roots is removed from the soil.

There are four types of peanuts grown commercially in the U.S.: runner, Virginia, Valencia, and Spanish. The runner type is the primary commercial peanut raised and is used mainly for peanut butter. The Virginia type is mainly used for gourmet snacks. The Valencia type are sweet-flavoured peanuts that are usually roasted and sold in their shells or made into peanut butter. Spanish peanuts have a red skin and pronounced nutty flavour due to higher oil content and are used to make peanut candies, shelled salted nuts, and peanut butter.

Peanuts are nutrient-dense, providing over 30 essential nutrients and phytonutrients, including niacin, folate, fiber, magnesium, vitamin E, manganese and phosphorus. Containing about 25% protein, peanuts are an excellent source of plant-based protein and offer an alternative to animal protein with similar digestibility for humans. This is important for the increasing demand for plant-based protein in the developed world. However, peanut is also an excellent source of protein in the developing world and is used to biofortify cereal-based and ready-to-use therapeutic foods. Peanut meal produced as a by-product from oil production can also be used to supply peanut protein for animal feed.

Peanuts are high in fat content, but importantly, are naturally free of trans-fats and sodium. According to the USDA, reference standards of raw, unroasted peanut seeds contain 49.24% total fat (USDA, 2015), with one ounce of raw peanuts containing approximately 12 grams of unsaturated fat, only two grams of saturated fat, and no trans-fat. According to the American peanut council, peanut fat profile contains about 50% monounsaturated fatty acids, 33% paraformaldehyde and 14% saturated fatty acids (Ayra, S. S. et al., J Food Sci Tecnol 53:31-41 (2016)). In addition, it has been reported that the fat content of peanuts ranges from 44%-56%, mainly consisting of mono- and poly-unsaturated fat which is mostly made up of oleic acid and linoleic acid (Arnarson A., Healthline (healthline.com) (May 7, 2019)).

Because peanuts serve as an excellent functional food, combining a good protein source with a host of other beneficial compounds, including fatty acids, vitamins and minerals, and antioxidants, organizations like the World Health Organization, UNICEF, Project Peanut Butter, and Doctors Without Borders have used peanut products to help save malnourished children in developing countries. Thus, improvement of the factors that indicate and/or affect both the food quality of peanuts and the peanut harvest is of considerable importance to the worldwide peanut processing and manufacturing community.

A continuing goal of peanut plant breeders is to develop stable, high yielding peanut cultivars that are agronomically sound and which meet market demands for products, such as shelled or unshelled roasted nuts, peanut butter, oil, peanut flour, and biodiesel, as well as non-food products, livestock feed, fuel, mulch, and manufacturing particle board or fertilizer. To accomplish this goal, the peanut breeder must select and develop peanut plants that have the traits that result in superior cultivars. There are numerous steps in the development of any novel, desirable plant germplasm. Plant breeding begins with the analysis, definition of problems and weaknesses of the current germplasm, the establishment of program goals, and the definition of specific breeding objectives. The next step is selection of germplasm that possesses the traits to meet the program goals. The goal is to combine in a single cultivar or hybrid an improved combination of desirable traits from the parental germplasm. These important traits may include improved flavor, higher yield, larger kernels, high oleic acid, high protein percentage, improved color, resistance to diseases and insects, tolerance to drought and heat, and better agronomic quality.

Methods for producing novel peanut lines through selection are known in the art. Each of the following references is incorporated in its entirety, herein, by reference: Moore, K. M. et al., J. Heredity 80 (3): 252 (1989); Norden, A. J., Peanuts, Culture and Uses. Am. Peanut Res. And Educ. Soc., Stillwater, Okla. (C. T. Wilson ed. 1973); Norden, A. J. in Hybridization of Crop Plants (H. H. Hadley ed. 1980); Norden, A. J., et al., Breeding of the cultivated peanut in Peanut Science and Technology, (H. E. Pattee ed. 1992); Norden, A. J. et al., Florida Agr. Res. 3:16-18 (1984); Knauft, D. A. et al., Peanut, Peanut Principles of Cultivar Development, 2:346-384 (Walter R. Fehr ed. 1987).

### SUMMARY OF THE INVENTION

The present invention provides a novel peanut cultivar designated IPG 110 and deposited under the terms of the Budapest Treaty with the Bigelow Provasoli-Guillard National Center for Marine Algae and Microbiota (NCMA) as Accession Number 202412003. The invention encompasses the seeds, plants, and plant parts of peanut cultivar IPG 110, as well as plants with essentially all of the physiological and morphological characteristics of IPG 110.

This invention also provides methods for producing a peanut plant by planting a plurality of seeds or by crossing peanut IPG 110 with itself, another peanut line, or a plant of a different species. Any plant breeding methods using peanut cultivar IPG 110 are part of this invention, including selfing, backcrosses, hybrid production, and crosses to populations. All plants and seeds produced using peanut cultivar IPG 110 as a parent are within the scope of this invention, including gene-converted seeds and plants of IPG 110. Methods for introducing a gene conversion, transgene, edited gene, and mutated gene, or gene element, into IPG 110 (i.e., either through traditional breeding, genetic engineering, gene editing, or mutagenesis) are also provided herein.

In still another aspect, the present invention provides regenerable cells for use in cell or tissue culture of peanut plant IPG 110, as well as peanut plants regenerated from these cultures.

### Definitions

To provide a clear and consistent understanding of the specification and claims, the following definitions are provided:

Allele. An allele is any of one or more alternative form of a gene, all of which relate to one trait or characteristic. In a diploid cell or organism, the two alleles of a given gene occupy corresponding loci on a pair of homologous chromosomes.

Alter. The utilization of up-regulation, down-regulation, or gene silencing.

Arachis hypogea L. The domesticated peanut, or groundnut, is an amphidiploid species in the legume or “bean” family and is an annual herbaceous plant.

Backcrossing. A process in which a breeder repeatedly crosses hybrid progeny back to a parental line. For example, a first generation (F₁) hybrid may be crossed with one of the parental lines used to produce the F₁ hybrids to generate a BC₁. Additional generational backcrosses to the recurrent parental line produce BC₂, BC₃, BC₄, BC₅, BC₆, BC₇, BC₈, BC₉, and BC₁₀ generations.

Breeding. The genetic manipulation of living organisms.

Cell. As used herein, this term includes isolated cells, cells grown in tissue culture, and cells that comprise a plant or plant part.

Cotyledon. A cotyledon is a type of seed leaf. The cotyledon contains the food storage tissues of the seed.

Chlorosis. Used to describe a reduced amount of chlorophyll resulting in light or yellow colored leaves.

Concentric chlorotic ring-spots. Light or dark areas on the leaf in the form concentric circles, ovals, or similar shape not necessarily symmetrical or uniform in appearance.

Diploid. A cell or organism having two sets of chromosomes.

Doubled haploid. A cell or organism comprising a doubled set of haploid chromosomes.

Embryo. The plant embryo is the part of a seed or bud that contains the earliest forms of the new plant's roots, stem and leaves.

Essentially all of the physiological and morphological characteristics. A plant having “essentially all the physiological and morphological characteristics” of the cultivar exhibits the characteristics of the cultivar with the exception of any characteristics derived from a converted gene, transgene, edited gene, mutated gene, or somaclonal variant.

F #. Denotes a filial generation, wherein the #is the generation number. For example, F₁ is the first filial generation.

Gene. Refers to a unit of inheritance corresponding to a distinct sequence of DNA or RNA nucleotides that form part of a chromosome. A gene may encode a polypeptide or a nucleic acid molecule that has a function in the cell or organism.

Gene-converted. Describes a plant wherein essentially all of the desired morphological and physiological characteristics of a parental cultivar are maintained with the exception of a single trait that was transferred into the cultivar via breeding (e.g., backcrossing), genetic engineering, gene-editing, or mutagenesis.

Gene mutation. A cell that includes a modified polynucleotide added to or modified within its genome compared to a non-genome mutated cell of the same type. In some cases, a non-genome mutated cell is a wild-type cell. The gene mutation may occur naturally or be induced by the hand of man via (e.g., mutagenesis).

Gene Silencing. The interruption or suppression of the expression of a gene at the level of transcription or translation.

Genetically modified. As used herein, the terms “genetically engineered” and “genetically modified” and are used interchangeably and refer to a prokaryotic or eukaryotic cell whose cellular nucleic acid, whether endogenous and/or exogenous, has been genetically modified or engineered using biotechnology techniques (e.g., transformation, genome editing, RNA interference, gene silencing).

Genome edited. A cell that includes an exogenous, heterologous, recombinant, synthetic, and/or otherwise modified polynucleotide added to or altered within its genome compared to a non-genome edited cell of the same type. In some cases, a non-genome edited cell is a wild-type cell.

Genotype. Refers to the genetic constitution of a cell or organism.

Habit. This refers to the physical appearance of a plant. In peanuts, it can be prostrate, decumbent, semi-erect, or erect.

Haploid. A cell or organism having a single set of unpaired chromosomes.

Herbicide-tolerant. Used interchangeably with the term “herbicide-resistant” to indicate that a plant or part thereof is capable of growing in the presence of an amount of herbicide that normally causes growth inhibition or phytotoxicity in a non-herbicide-tolerant (e.g., a wild-type) plant or part thereof. Levels of herbicide that normally inhibit growth of a non-tolerant plant are known and readily determined by those skilled in the art. Examples include the quantity of herbicide or rate of application recommended by herbicide manufacturers. The maximum level or rate of herbicide application is the amount of herbicide that would normally inhibit the growth or cause phytotoxicity of a non-herbicide tolerant plant.

Hilum. This refers to the scar left on the seed that marks the place where the seed was attached to the pod prior to the seed being harvested.

Hybrid. Refers to the offspring or progeny of genetically dissimilar plant parents or stock produced as the result of controlled cross-pollination as opposed to a non-hybrid seed produced as the result of natural pollination.

Hypocotyl. A hypocotyl is the portion of an embryo or seedling between the cotyledons and the root. Therefore, it can be considered a transition zone between shoot and root.

LB/A. Pounds per Acre. The seed yield in pounds/acre is the actual yield of the peanut at harvest.

Leaflets. These are part of the plant shoot, and they manufacture food for the plant by the process of photosynthesis.

Leaf petiole. The small stalk attaching the leaf blade to the stem.

Leaf spots. A spot on a leaf usually resultant from infection; can be either chlorotic or necrotic and may be ringed, referred to as a ring-spot.

Linkage. Refers to a phenomenon wherein alleles on the same chromosome tend to segregate together more often than expected by chance if their transmission was independent.

Linkage disequilibrium. Refers to a phenomenon wherein alleles tend to remain together in linkage groups when segregating from parents to offspring, with a greater frequency than expected from their individual frequencies.

Locus. A locus confers one or more traits such as, for example, male sterility, herbicide tolerance, insect resistance, disease resistance, waxy starch, modified fatty acid metabolism, modified phytic acid metabolism, modified carbohydrate metabolism, and modified protein metabolism. The trait may be, for example, conferred by a naturally occurring gene introduced into the genome of the cultivar by backcrossing, a natural or induced mutation, or a transgene introduced through genetic transformation techniques. A locus may comprise one or more alleles integrated at a single chromosomal location.

Maturity Date. Plants are considered mature when 95% of the pods have reached their mature color. The number of days are calculated either from August 31 or from the planting date.

Maturity Group. This refers to an agreed-upon industry division of groups of peanut cultivars based on length of time needed to reach commercial harvest maturity (prior to digging), which is generally considered to be approximately 70% of pods that have black or brown coloration of the mesocarp. These categories can be generally grouped as “early”, reaching harvest maturity by approximately 110-130 days after planting (DAP); “medium”, reaching maturity by approximately 130-145 DAP; and “late”, requiring more than 145 DAP to reach harvest maturity.

Mottling. Abnormal coloration on plants, usually a sign of disease or malnutrition.

Oil or Oil Percent. Peanut seeds contain a considerable amount of oil. Oil is measured by NIR spectrophotometry and is reported as a percentage basis.

Peanut. The seed of a peanut plant, also known as earthnuts, ground nuts, goober peas, monkey nuts, pygmy nuts, and pig nuts.

Peanut flour. Flour high in protein, often used as a gluten-free solution.

Peanut Rx. An index designed to help growers approximate the magnitude of the risk that they face from diseases in the coming season.

Pedigree. Refers to the lineage or genealogical descent of a plant.

Pedigree Distance. Relationship among generations based on their ancestral links as evidenced in pedigrees. May be measured by the distance of the pedigree from a given starting point in the ancestry.

Percent Identity. Percent identity as used herein refers to the comparison of the homozygous alleles of two peanut varieties. Percent identity is determined by comparing a statistically significant number of the homozygous alleles of two developed varieties. For example, a percent identity of 90% between peanut cultivar 1 and peanut cultivar 2 means that the two varieties have the same allele at 90% of their loci.

Percent Similarity. Percent similarity as used herein refers to the comparison of the homozygous alleles of a peanut cultivar such as peanut cultivar IPG 517 with another plant, and if the homozygous allele of peanut cultivar IPG 517 matches at least one of the alleles from the other plant, then they are scored as similar. Percent similarity is determined by comparing a statistically significant number of loci and recording the number of loci with similar alleles as a percentage. A percent similarity of 90% between peanut cultivar IPG 517 and another plant means that peanut cultivar IPG 517 matches at least one of the alleles of the other plant at 90% of the loci.

Plant. As used herein, the term “plant” includes plant cells, plant protoplasts, and plant cell tissue cultures from which peanut plants can be regenerated; plant calli, plant clumps, meristematic cells, and plant cells that are intact in plants; and parts of plants, such as embryos, pollen, ovules, seeds, flowers, glumes, panicles, leaves, stems, shoots, suckers, internodes, buds, roots, root tips, anthers, and pistils.

Plant Height. Plant height is taken from the top of the soil to the top node of the plant and is measured in centimeters.

Plant parts. Includes, without limitation, protoplasts, leaves, stems, internodes, buds, roots, root tips, anthers, pistils, seed, grain, nut, peanut, embryo, pollen, ovules, cotyledon, hypocotyl, pod, flower, shoot, tissue, petiole, pedicel, pistils, cells, meristematic cells, and the like.

Pod. This refers to the fruit of a peanut plant. It consists of the hull or shell (pericarp) and the peanut seeds.

Progeny. As used herein, includes an F₁ peanut plant produced from the cross of two peanut plants where at least one plant includes peanut cultivar IPG 517 and progeny further includes, but is not limited to, subsequent generations.

Protein Percent. Peanut seeds contain a considerable amount of protein. Protein is generally measured by NIR spectrophotometry and is reported as percentage basis. Types of protein may include but are not limited to cupin and prolamin storage proteins. Examples of storage proteins include but are not limited to Ara h 1, Ara h 2, Ara h 3, Ara h 4, Ara h 5, Ara h 6, Ara h7, Ara h 8, and Ara h 9.

Quantitative Trait Loci. Quantitative Trait Loci (QTL) refers to genetic loci that control to some degree, numerically representable traits that are usually continuously distributed.

Regeneration. Regeneration refers to the development of a plant from tissue culture.

Resistance. The intrinsic ability of a plant to tolerate or withstand external stimuli or pressure (e.g., pressure from diseases, insects, herbicides, heat, cold, drought, salinity, abiotic, and biotic stressors).

Seed Oleic Acid Content. The percentage of oleic acid in the peanut, as measured by gas chromatography or near-infrared spectrometry. High seed oleic acid content is equal to a range of approximately 70-80%. Normal seed oleic acid content is equal to a range of approximately 40-50%.

Seeds. Includes seeds and plant propagules of all kinds including, but not limited to, true seeds, seed pieces, suckers, corms, bulbs, fruit, tubers, grains, nuts, peanuts, cuttings, cut shoots and the like. However, in preferred embodiments, it refers to true seeds.

Single gene converted. Single gene converted or conversion plant refers to plants which are developed by traditional breeding methods (e.g., backcrossing), via genetic engineering, gene-editing, or mutagenesis, wherein essentially all of the desired morphological and physiological characteristics of a line are recovered in addition to the single gene transferred into the line via the breeding technique, genetic engineering, gene-editing, or mutagenesis.

Subline. Although peanut cultivar IPG 110 contains substantially fixed genetics, is phenotypically uniform with no off-types expected, there still remains a small proportion of segregating loci either within individuals or within the population as a whole. A breeder of ordinary skill in the art may fix these loci by making them more uniform in order to optimize the performance of the cultivar. One example of this type of approach is described in the “breeding bias” methods described in U.S. Pat. No. 5,437,697 and may be utilized by a breeder of ordinary skill in the art to further purify the cultivar in order to increase its yield. By sublining in this manner, no crosses to a different cultivar are made, and so a new genetic cultivar is not created and the overall genetic composition of the cultivar remains essentially the same.

Thrips. Tiny insects which carry and transmit disease. The two most common species being Tobacco thrips (Frankliniella fusca) and Western flower thrips (Frankliniella occidentalis). They are also referred to as thunderflies, thenderbugs, storm flies, thrunderblights, and corn lice.

Tomato Spotted Wilt Tospovirus (TSWV). A class V virus having a single stranded RNA genome with negative polarity found within the family Bunyaviridae. TSWV is an arbovirus usually vectored by thrips and is common in warm climates such as Asia, America, Europe and Africa.

Total Sound Mature Kernels (TSMK). The percentage of SMK (sound mature kernels) riding a screen with 0.64 by 1.91 cm slots plus sound splits (i.e. the sum of SMK and sound splits). TSMK is the commercially-standardized metric for evaluating grade or quality of peanuts and thereby directly impacts sale-ability.

Trait. Refers to an observable and/or measurable characteristic of an organism.

Transgene. Any DNA sequences, whether from a different species or from the same species, which are introduced into the genome using transformation or various breeding methods are referred to herein collectively as “transgenes.”

Variety. Synonymous with cultivar, a substantially homozygous peanut line which may comprise minor modifications thereof, including but not limited to resulting from sublining, creation of a doubled haploid line, a locus conversion, a mutation, a transgene, an edited gene, or a somaclonal variant, but which otherwise retains the overall genetics of the peanut line.

Wild-type. When made in reference to a gene, “wild-type” refers to a functional gene common throughout a plant population and, thus, arbitrarily designated the “normal” or “wild-type” form of the gene.

Yield (Pounds/Acre). The yield in pounds/acre is the actual yield of the peanut at harvest.

### DETAILED DESCRIPTION OF THE INVENTION

The present invention provides a novel peanut cultivar designated IPG 110 and deposited under the terms of the Budapest Treaty with the Bigelow Provasoli-Guillard National Center for Marine Algae and Microbiota (NCMA) as Accession Number 202412003. The invention encompasses both the seeds of this cultivar and plants grown from these seeds. The invention further encompasses any peanut plant having all or essentially all of the physiological and morphological characteristics peanut cultivar IPG 110.

As used herein, the term plant includes plant cells, plant protoplasts, plant cell tissue cultures from which peanut plants can be regenerated, plant calli, plant clumps, and parts of plants, such as leaves, stems, internodes, buds, roots, root tips, anthers, pistils, seed, nut, peanut, embryo, pollen, ovules, cotyledon, hypocotyl, pod, flower, shoot, tissue, petiole, pedicel, pistil, cells, meristematic cells, and the like.

### Development and Characterization of Peanut Cultivar IPG 110 (Experimental Number (18-4-0110)

Peanut cultivar IPG 110 is a red-seeded, normal oleic, Valencia-type cultivar with an upright growth habit and early maturity. IPG 110 is also characterized by its high protein percentage (approximately 30%) when compared to cultivars of similar maturity.

Some of the selection criteria used in developing IPG 110 include the following traits: pod yield, pod conformation and appearance, grade, seed size, number of kernels per pod, seed coat color, protein percentage, fatty acid composition, oil content, oleic acid content, flavor profile of roasted kernels, disease resistance, seedling emergence, early-season vigor, disease tolerance, herbicide tolerance, maturity (early), late season plant intactness. In addition, the cultivar has been phenotypically selected for uniformity of plant type, pod type, and stability, as described in the following cultivar description information. Off-type plants (for either plant architecture or pod type or other phenotypic variation) have been rogued at various stages of cultivar development, and the cultivar has been increased by self-pollination with continued observation of and selection for uniformity.

Table 1 describes the developmental timeline for cultivar IPG 110, while Table 2 outlines the phenotypic characteristics of cultivar IPG 110. The results of 2019 Yoakum County, Texas yield trials for cultivar IPG 110 and check cultivars are presented in Table 3. The results of 2020-2023 Terry County, Texas yield trials for cultivar IPG 110 and check cultivars are presented in Tables 4-7. Additional plant phenotypic data collected from the 2020-2023 Terry County, Texas trials for cultivar IPG 110 and check cultivars is presented in Tables 8-12. Stability and uniformity observations collected in Yoakum County, Texas and Terry County, Texas by year and location are reported for cultivar IPG 110 in Table 13.

Nutritional composition analysis of IPG 110 revealed this cultivar as having elevated protein content in the mature kernels (approximately 29.4-30.5%). This value represents an approximate 24-29% increase in protein content over the current industry average for Southeastern runner-type Jumbo kernels (23.6%) and an approximately 31-36% increase over that of Georgia-06G (U.S. PVP 200700208) seed (22.4%), with Georgia-06G the most-grown peanut cultivar in the U.S. IPG 110 ranked first for protein percentage in Terry County, Texas trials for three consecutive years (2020-2022), including: all 9 check cultivars in 2020 (Table 7); all 13 check cultivars in 2021 (Table 9); and all 6 check cultivars in 2022 (Table 10). IPG 110 exhibited comparable total sound mature kernels (TSMK) when compared to several normal oleic checks in the aforementioned trials (Tables 3-6). IPG 110 is also characterized by its tall mainstem height, and long, wide leaves (Tables 11-12).

TABLE 1
IPG 110 development timeline

| Year | Program stage |
|---|---|
| 2023 | Terry County, Texas Yield Test (Tables 7 and 12); Evaluated IPG 110 for |
| | stability and uniformity (Table 13) |
| 2022 | Terry County, Texas Yield Trials (Tables 6, 8, and 10-11); Evaluated IPG 110 |
| | for stability and uniformity (Table 13) |
| 2021 | Terry County, Texas Yield Trial (Table 5 and 9); Evaluated IPG 110 for |
| | stability and uniformity (Table 13) |
| 2020 | Terry County, Texas Yield Test (Table 4); Evaluated IPG 110 for stability and |
| | uniformity (Table 13) |
| 2019 | Yoakum County, Texas Yield Test (Table 3); Evaluated IPG 110 for stability |
| | and uniformity (Table 13) |
| 2018 | F_{2:7} seeds were planted in two-row field plots in Texas. IPG 110 (Experimental |
| | number 18-4-0110) was selected as an F_{2:7} uniform line and bulk-harvested for |
| | replicated yield testing in 2019. |
| 2017 | F_{2:6} seeds were planted in two-row field plots. Individual plants were selected |
| | and self-pollinated to generate seed for planting the following year. |
| 2016 | F_{2:5} seeds were planted in two-row field plots. Individual plants were selected |
| | and self-pollinated to generate seed for planting the following year. |
| 2015 | F_{2:4} seeds were planted in two-row field plots. Individual plants were selected |
| | and self-pollinated to generate seed for planting the following year. |
| 2014 | F_{2:3} seeds were planted in two-row field plots. Individual plants were selected |
| | and self-pollinated to generate seed for the following year. |
| 2013 | F₂ seeds were grown in a field in Worth County, Georgia. Individual F₂ plants |
| | were selected and self-pollinated to generate seed of F₂ derived families for |
| | planting the following year. |
| 2012 | F₁ plants were grown in a field in Worth County, Georgia and self-pollinated. |
| | All viable F₁ plants that produced seed were bulk-harvested. |
| 2011 | Bi-parental cross of ‘SPZ 494-2’ (PI 502111) (female parent, unpatented)/ |
| | ‘WT08-0085’ (male parent, unpatented, unreleased) |

Peanut cultivar IPG 110 is similar to its female parent, peanut cultivar SPZ 494-2, in that both are characterized by normal seed oleic acid content, an upright growth habit, and early maturity. IPG 110 exhibits distinct phenotypic differences compared to WT08-0085, including but not limited to: 1) seed oleic acid content: IPG 110 exhibits normal seed oleic acid content, 5 while WT08-0085 exhibits high seed oleic acid content.

Peanut cultivar IPG 110 is similar to peanut cultivar Valencia C in that both cultivars are characterized by normal-oleic acid seed content and both have similar total sound mature kernels (TSMK) (Tables 3-6). However, peanut cultivar IPG 110 differs from cultivar Valencia C in pod yield, with IPG 110 being lower yielding compared to Valencia C (Table 3). IPG 110 matures earlier than peanut cultivar IPG 1288, having 115 days to maturity compared to IPG 1288's 125 days to maturity (Table 2).

TABLE 2
IPG 110 cultivar description information.

| Category | Parameter | Description | |
|---|---|---|---|
| Plant | Growth habit: | Erect | |
| | Flowering on the main stem: | Present | |
| | Branching pattern: | Sequential | |
| | Branching: | Sparse | |
| | Lateral branch length: | 37 | cm |
| | Mainstem height: | 38 | cm |
| Maturity | Region: | Texas, United States | |
| | Number of days to maturity: | 115 | |
| | Days earlier than comparison | 10 | |
| | peanut cultivar IPG 1288: | | |
| | Days later than comparison | 0 | |
| | peanut cultivar Valencia C: | | |
| Leaves | Arrangement: | Opposite, pinnate, | |
| | | and tetrafoliate | |
| | Leaflet length: | 4.4 | cm |
| | Leaflet width: | 2.0 | cm |
| | Leaflet length/width ratio: | 2.2 | |
| Flower | Color: | Yellow | |
| | Days to flowering: | 25 to 35; indeterminate | |
| | Arrangement: | Axillary; from leaf axil | |
| Pod | Shape: | Oblong, indehiscent legume | |
| | Length: | 34 | mm |
| | Diameter: | 13 | mm |
| | Number of seeds per pod: | 2, 3, or 4 | |
| | Pod yield (lb/A): | 2000 to 3500 | |
| | Surface: | Glabrous | |
| | Constriction: | Shallow | |
| | Beak: | Inconspicuous | |
| Seed | Coat color: | Red | |
| | Coat surface: | Smooth | |
| | Shape: | Elongated-Slender | |
| | Grams per 100 seeds | 40.0 | |
| | (8% moisture): | | |
| | Length: | 13 | mm |
| | Width: | 8 | mm |

TABLE 3
Pod yield, grade, and seed size distribution results of IPG
110 and three cultivars in Yoakum County, Texas in 2019.

| | | | | | | Seed |
|---|---|---|---|---|---|---|
| | Pod | | | | | Oleic |
| | Yield | TSMK^{a} | Jumbo^{b} | Medium^{c} | No. 1^{d} | Acid |
| Entry | lb/A | % | | | | Content^{e} |
| IPG 1288 | 3020 | 73 | 41 | 40 | 4 | High |
| IPG 274 | 2982 | 73 | 40 | 39 | 6 | High |
| Valencia C | 1425 | 63 | 0 | 54 | 31 | Normal |
| IPG 110 | 1327 | 64 | 0 | 38 | 46 | Normal |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm screen.
^{c}Whole peanut kernels that did not pass through a 7.1-mm × 19.0-mm screen.
^{d}Whole peanut kernels that did not pass through a 5.9-mm × 19.0-mm screen.
^{e}Seed oleic acid content, as measured by gas chromatography, where “Normal” values range between 40-50%, and “High” values range between 70-80%.

TABLE 4
Pod yield, grade, and seed size distribution results of IPG 110
and three Valencia cultivars in Terry County, Texas in 2020.

| | | | | | | Seed |
|---|---|---|---|---|---|---|
| | Pod | | | | | Oleic |
| | Yield | TSMK^{a} | Jumbo^{b} | Medium^{c} | No. 1^{d} | Acid |
| Entry | lb/A | % | | | | Content^{e} |
| IPG 1288 | 2149 | 69 | 26 | 40 | 11 | High |
| IPG 274 | 1878 | 69 | 27 | 40 | 12 | High |
| Valencia C | 1439 | 60 | 1 | 51 | 33 | Normal |
| IPG 110 | 995 | 58 | 0 | 40 | 40 | Normal |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm screen.
^{c}Whole peanut kernels that did not pass through a 7.1-mm × 19.0-mm screen.
^{d}Whole peanut kernels that did not pass through a 5.9-mm × 19.0-mm screen.
^{e}Seed oleic acid content, as measured by gas chromatography, where “Normal” values range between 40-50%, and “High” values range between 70-80%.

TABLE 5
Pod yield, grade, and seed size distribution results of IPG 110
and three Valencia cultivars in Terry County, Texas in 2021.

| | | | | | | Seed |
|---|---|---|---|---|---|---|
| | Pod | | | | | Oleic |
| | Yield | TSMK^{a} | Jumbo^{b} | Medium^{c} | No. 1^{d} | Acid |
| Entry | lb/A | % | | | | Content^{e} |
| IPG 274 | 4012 | 78 | 59 | 22 | 4 | High |
| IPG 1288 | 3849 | 78 | 60 | 26 | 4 | High |
| Valencia C | 3461 | 71 | 8 | 67 | 17 | Normal |
| IPG 110 | 2854 | 69 | 2 | 57 | 32 | Normal |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm screen.
^{c}Whole peanut kernels that did not pass through a 7.1-mm × 19.0-mm screen.
^{d}Whole peanut kernels that did not pass through a 5.9-mm × 19.0-mm screen.
^{e}Seed oleic acid content, as measured by gas chromatography, where “Normal” values range between 40-50%, and “High” values range between 70-80%.

TABLE 6
Pod yield, grade, and seed size distribution results of IPG 110
and two Valencia cultivars in Terry County, Texas in 2022.

| | | | | | | Seed |
|---|---|---|---|---|---|---|
| | Pod | | | | | Oleic |
| | Yield | TSMK^{a} | Jumbo^{b} | Medium^{c} | No. 1^{d} | Acid |
| Entry | lb/A | % | | | | Content^{e} |
| IPG 1288 | 3849 | 71 | 26 | 37 | 16 | High |
| IPG 110 | 3215 | 69 | 0 | 44 | 35 | Normal |
| Valencia C | 2839 | 68 | 3 | 51 | 30 | Normal |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm screen.
^{c}Whole peanut kernels that did not pass through a 7.1-mm × 19.0-mm screen.
^{d}Whole peanut kernels that did not pass through a 5.9-mm × 19.0-mm screen.
^{e}Seed oleic acid content, as measured by gas chromatography, where “Normal” values range between 40-50%, and “High” values range between 70-80%.

TABLE 7
Pod yield, grade, and seed size distribution results of IPG
110 and one Valencia cultivar in Terry County, Texas in 2023.

| | | | | | | Seed |
|---|---|---|---|---|---|---|
| | Pod | | | | | Oleic |
| | Yield | TSMK^{a} | Jumbo^{b} | Medium^{c} | No. 1^{d} | Acid |
| Entry | lb/A | % | | | | Content^{e} |
| IPG 1288 | 3012 | 73 | 41 | 34 | 10 | High |
| IPG 110 | 1526 | 62 | 2 | 40 | 35 | Normal |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm screen.
^{c}Whole peanut kernels that did not pass through a 7.1-mm × 19.0-mm screen.
^{d}Whole peanut kernels that did not pass through a 5.9-mm × 19.0-mm screen.
^{e}Seed oleic acid content, as measured by gas chromatography, where “Normal” values range between 40-50%, and “High” values range between 70-80%.

TABLE 8
Nutritional content of IPG 110, seven peanut cultivars
and two advanced lines in Terry County, Texas in 2020^{a}.

| | Protein | | Saturated | Total |
|---|---|---|---|---|
| | Content | Oleic:Linoleic | Fat Content | Fat Content |
| Entry | % | Ratio | % | |
| IPG 110 | 30.00 | 1.00 | 18.97 | 40.73 |
| 19-4-0103 | 29.58 | 0.94 | 18.22 | 41.89 |
| 17-4-0506 | 27.92 | 1.00 | 18.39 | 42.34 |
| IPG 914 | 24.55 | 17.82 | 12.96 | 41.35 |
| IPG 3628 | 24.27 | 18.61 | 13.20 | 42.51 |
| IPG QR-14 | 24.19 | 15.10 | 13.30 | 41.63 |
| IPG 1288 | 23.58 | 17.35 | 14.38 | 44.19 |
| IPG 464 | 23.18 | 29.84 | 12.44 | 44.72 |
| IPG 2309 | 22.62 | 8.39 | 14.25 | 33.70 |
| IPG 913 | 21.97 | 1.84 | 17.95 | 48.14 |

^{a}Analyses conducted by Dr. Julie Marshall and BRL Analytical Services, Lubbock, TX.

TABLE 9
Nutritional content of IPG 110, eleven peanut cultivars,
and two advanced lines in Terry County, Texas in 2021^{a}.

| | |
|---|---|
| Protein | Total Fat |
| Content | Content |

| | |
|---|---|
| Entry | % |

| | | |
|---|---|---|
| IPG 110 | 30.5 | 41.64 |
| 19-4-0103 | 30.1 | 41.10 |
| 17-4-0506 | 29.9 | 37.78 |
| IPG QR-14 | 24.2 | 45.58 |
| IPG 914 | 24.9 | 44.72 |
| IPG 464 | 26.9 | 41.17 |
| IPG 3628 | 24.1 | 42.20 |
| IPG 274 | 25.3 | 43.41 |
| IPG 1288 | 24.8 | 44.80 |
| ACI 883 | 24.6 | 42.96 |
| ACI 789 | 25.0 | 46.94 |
| ACI 442 | 26.6 | 40.36 |
| ACI 236 | 24.9 | 46.48 |
| IPG 913 | 22.4 | 46.68 |

^{a}Analyses conducted by Dr. Julie Marshall and BRL Analytical Services, Lubbock, TX.

TABLE 10
Nutritional content of IPG 110, four peanut cultivars,
and two advanced lines in Terry County, Texas in 2022^{a}.

| | Protein | | Saturated | Total |
|---|---|---|---|---|
| | Content | Oleic:Linoleic | Fat Content | Fat Content |
| Entry | % | Ratio | % | |
| IPG 110 | 29.4 | 0.96 | 19.21 | 39.88 |
| 19-4-0103 | 29.3 | 0.93 | 19.40 | 41.71 |
| 17-4-0506 | 29.0 | 0.93 | 18.25 | 41.94 |
| Georgia-09B | 24.3 | 20.72 | 14.27 | 39.44 |
| Georgia-06G | 22.4 | 1.53 | 18.67 | 44.62 |
| IPG 913 | 19.2 | 1.59 | 17.58 | 46.38 |
| IPG 2309 | 19.1 | 6.70 | 15.07 | 30.33 |

^{a}Analyses conducted by Dr. Julie Marshall and BRL Analytical Services, Lubbock, TX.

TABLE 11
Leaflet length and width, mainstem height, and
lateral branch length of IPG 110 and seventeen
(17) cultivars in Terry County, Texas in 2022.

| | | | | | Lateral |
|---|---|---|---|---|---|
| | Leaflet | Leaflet | Leaflet | Mainstem | Branch |
| | Length^{a} | Width^{a} | Length/Width | Height | Length |
| Entry | cm | | Ratio | cm | |
| ACI 442 | 4.6 | 2.0 | 2.30 | 14 | 37 |
| ACI 789 | 4.2 | 1.9 | 2.21 | 16 | 35 |
| AT-9899 | 4.4 | 1.9 | 2.32 | 13 | 34 |
| Georgia-06G | 4.1 | 1.7 | 2.41 | 14 | 35 |
| Georgia-09B | 3.4 | 1.6 | 2.13 | 14 | 32 |
| Georgia-16HO | 4.2 | 1.8 | 2.33 | 11 | 33 |
| IPG 110 | 4.6 | 2.1 | 2.19 | 19 | 36 |
| IPG 464 | 4.5 | 2.0 | 2.25 | 13 | 37 |
| IPG 517 | 4.0 | 1.6 | 2.50 | 16 | 32 |
| IPG 914 | 4.2 | 1.5 | 2.80 | 15 | 37 |
| IPG 1288 | 4.5 | 2.1 | 2.14 | 13 | 42 |
| IPG 2309 | 4.9 | 2.0 | 2.45 | 18 | 43 |
| IPG 3628 | 4.3 | 1.9 | 2.26 | 16 | 37 |
| IPG QR-14 | 4.0 | 1.8 | 2.22 | 14 | 32 |
| TUFRunner ™ | 4.1 | 1.8 | 2.28 | 17 | 35 |
| ‘297’ | | | | | |
| TUFRunner ™ | 4.0 | 1.7 | 2.35 | 16 | 40 |
| ‘727’ | | | | | |
| Valencia C | 4.7 | 2.1 | 2.24 | 19 | 35 |

^{a}Leaflet data were collected from the basal leaflet of the first fully-formed leaf at the top of the mainstem.

TABLE 12
Leaflet length and width, mainstem height, and lateral branch length
of sixteen (16) cultivars in Terry County, Texas in 2023.

| | | | | | Lateral |
|---|---|---|---|---|---|
| | Leaflet | Leaflet | Leaflet | Mainstem | Branch |
| | Length^{a} | Width^{a} | Length/Width | Height | Length |
| Entry | cm | | Ratio | cm | |
| ACI 442 | 4.1 | 1.6 | 2.56 | 17 | 34 |
| AT-9899 | 3.6 | 1.4 | 2.57 | 17 | 32 |
| Georgia-06G | 4.1 | 1.7 | 2.41 | 14 | 35 |
| Georgia-09B | 3.4 | 1.4 | 2.43 | 15 | 29 |
| Georgia-16HO | 4.2 | 1.8 | 2.28 | 11 | 33 |
| IPG 110 | 4.4 | 2.0 | 2.20 | 38 | 37 |
| IPG 517 | 4.0 | 1.5 | 2.67 | 17 | 31 |
| IPG 913 | 4.1 | 1.8 | 2.28 | 14 | 32 |
| IPG 914 | 4.2 | 1.5 | 2.80 | 15 | 37 |
| IPG 1288 | 3.5 | 1.4 | 2.50 | 17 | 34 |
| IPG 2309 | 4.2 | 1.6 | 2.63 | 20 | 39 |
| IPG 3628 | 3.7 | 1.4 | 2.64 | 17 | 33 |
| IPG QR-14 | 3.5 | 1.5 | 2.33 | 14 | 27 |
| Tamnut OL06 | 4.7 | 1.9 | 2.47 | 26 | 27 |
| TUFRunner ™ | 4.1 | 1.8 | 2.28 | 17 | 35 |
| ‘297’ | | | | | |
| TUFRunner ™ | 4.0 | 1.7 | 2.35 | 16 | 40 |
| ‘727’ | | | | | |

^{a}Leaflet data were collected from the basal leaflet of the first fully-formed leaf at the top of the mainstem.

TABLE 13
Crop years and locations of production and observation
of ‘IPG 110’ for stability and uniformity.

| Crop Year | Test Location | Plant Type^{a} | Pod Type^{b} |
|---|---|---|---|
| 2019 | Yoakum Co., TX | Uniform | Uniform |
| 2020 | Terry Co., TX | Uniform | Uniform |
| 2021 | Terry Co., TX | Uniform | Uniform |
| 2022 | Terry Co., TX | Uniform | Uniform |
| 2023 | Terry Co., TX | Uniform | Uniform |
| 2024 | Terry Co., TX | Uniform | Uniform |

^{a}Plant type is determined based on phenotypic observation of plant architecture. A “Uniform” rating indicates the absence of obvious off-type plants.
^{b}Pod type is determined based on phenotypic observation of pod size and shape. A “Uniform” rating indicates the absence of obvious off-type pods.

TABLE 14
Kernel nutritional content of nineteen (19) peanut
cultivars in Terry County, Texas in 2024^{a}.

| | | |
|---|---|---|
| Protein | Total Fat | Oleic Acid |
| Content | Content | Content |

| | |
|---|---|
| Entry | % |

| | | | |
|---|---|---|---|
| IPG 110 | 31.3 | 39.46 | 40.59 |
| IPG 464 | 26.7 | 44.62 | 81.72 |
| ACI 442 | 26.1 | 45.28 | 81.10 |
| IPG 274 | 26.1 | 42.67 | 78.87 |
| Tifguard | 26.1 | 45.58 | 44.55 |
| ACI 236 | 26.0 | 41.79 | 76.27 |
| Bailey II | 25.8 | 41.48 | 81.75 |
| IPG 914 | 25.7 | 41.73 | 81.32 |
| OLin | 25.5 | 42.56 | 70.42 |
| Emery | 25.4 | 44.08 | 81.68 |
| Georgia-09B | 25.4 | 44.14 | 80.51 |
| IPG QR-14 | 25.0 | 44.00 | 81.64 |
| Georgia-06G | 25.3 | 48.11 | 48.55 |
| IPG 1288 | 25.3 | 42.64 | 77.91 |
| IPG 913 | 25.1 | 43.59 | 49.74 |
| IPG 517 | 24.6 | 43.76 | 81.08 |
| Georgia-16HO | 24.5 | 44.94 | 62.60 |
| IPG 3628 | 24.5 | 40.42 | 79.94 |
| TUFRunner ™ ‘297’ | 24.4 | 44.14 | 80.39 |

^{a}Analyses conducted by Dr. Julie Marshall and BRL Analytical Services, Lubbock, TX.

TABLE 15
Leaflet length and width, mainstem height, and
lateral branch length of IPG 110 and eleven (11)
cultivars in Terry County, Texas in 2024.

| | | | | | Lateral |
|---|---|---|---|---|---|
| | Leaflet | Leaflet | Leaflet | Mainstem | Branch |
| | Length^{a} | Width^{a} | Length/Width | Height | Length |
| Entry | cm | | Ratio | cm | |
| IPG 110 | 5.3 | 2.3 | 2.32 | 36 | 32 |
| ACI 442 | 5.7 | 2.2 | 2.63 | 19 | 30 |
| Bailey II | 5.0 | 2.1 | 2.40 | 22 | 28 |
| Georgia-09B | 4.1 | 1.8 | 2.32 | 19 | 25 |
| IPG 1288 | 4.8 | 1.9 | 2.59 | 19 | 35 |
| IPG 3628 | 4.5 | 2.0 | 2.22 | 21 | 25 |
| IPG 517 | 4.4 | 1.6 | 2.70 | 21 | 27 |
| IPG 913 | 4.2 | 1.9 | 2.20 | 20 | 25 |
| NM-310 | 5.2 | 2.4 | 2.18 | 37 | 37 |
| Schubert | 5.3 | 2.1 | 2.46 | 31 | 29 |
| Valencia C | 5.2 | 2.2 | 2.37 | 23 | 31 |

^{a}Leaflet data were collected from the basal leaflet of the first fully-formed leaf at the top of the mainstem.

### Methods

This present invention provides methods for producing peanut plants. In some embodiments, these methods involve crossing a first parent peanut plant with a second parent peanut plant wherein either the first or second parent peanut plant is a peanut plant of the cultivar IPG 110. Further, both first and second parent peanut plants may be peanut cultivar IPG 110. Self-pollinated plants of peanut cultivar IPG 110 are part of the invention, including repeated generations of self-pollinated plants of the invention or creation of doubled haploid plants of the invention. Still further, this invention also is directed to methods for producing a peanut cultivar IPG 110-derived peanut plant by crossing peanut cultivar IPG 110 with a second peanut plant and growing the progeny seed, wherein the crossing and growing steps may be repeated with the peanut cultivar IPG 110-derived plant from 0 to 7 times, or more. Thus, any such methods using the peanut cultivar IPG 110 are part of this invention: selfing, recurrent selection, pedigree breeding, backcrosses, hybrid production, crosses to populations, and the like. All plants produced using peanut cultivar IPG 110 as a parent are within the scope of this invention, including plants derived from peanut cultivar IPG 110, and doubled haploids of IPG 110, progeny of IPG 110, and plants derived from IPG 110. Advantageously, the peanut cultivar is used in crosses with different peanut cultivars to produce first generation (F₁) peanut seeds and plants with superior characteristics.

In one aspect, a IPG 110-derived peanut plant, a progeny plant, a genetically modified plant, a transformed plant, a mutated plant, a gene-edited plant, a regenerated plant, somaclonal variant, or other genetic variant is selected that has molecular markers, morphological characteristics, and/or physiological characteristics in common with IPG 110 (e.g., those listed in Table 2).

Particular markers used for these purposes are not limited to any particular set of markers but are envisioned to include any type of marker and marker profile which provides a means of distinguishing varieties for identification or selection purposes. Primers and PCR protocols for assaying these and other markers may be used for identification of peanut cultivar IPG 110, and plant parts and plant cells of peanut cultivar IPG 110. The genetic profile (i.e., genotype) may be used to identify a peanut plant produced through the use of peanut cultivar IPG 110; or to verify a pedigree for progeny plants or derivative plants produced through the use of peanut cultivar IPG 110. The genetic marker profile is also useful in breeding and developing backcross conversions. For example, a plant of cultivar IPG 110 comprising a single gene conversion, transgene, modified gene, edited gene, or genetic sterility factor, may be identified by having a molecular marker profile with a high percent identity to peanut cultivar IPG 110. Such a percent identity might be 95%, 96%, 97%, 98%, 99%, 99.5%, or 99.9% identical to peanut cultivar IPG 110. The genetic marker profile during conversion or modification may also be ascertained for purposes of recovering a higher percentage of the recurrent parent genome (i.e., during backcrossing) via measuring either percent identity or percent similarity.

Examples of molecular markers include: Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) (which are also referred to as Microsatellites), and Single Nucleotide Polymorphisms (SNPs).

In another aspect, IPG 110 may be self-pollinated or subjected to the process of creating doubled haploids, the processes of which fix or make homozygous residual heterozygous alleles at one or more loci in the IPG 110 genome. The resulting plants have all or essentially all of the physiological and morphological characteristics of IPG 110.

Further, this invention provides methods for introducing a desired trait into peanut cultivar IPG 110. This may be accomplished using traditional breeding methods, such as backcrossing (see Breeding Methods section below). Alternatively, the desired trait may be introduced by transforming the peanut cultivar with a transgene (see Transformation Methods section below), by mutagenizing a gene within the peanut's genome (see Mutagenesis Methods section below), or by editing a gene within the peanut's genome (see Gene Editing Methods section below). The transgenic, mutant, or edited cultivar produced by these methods may be crossed via traditional breeding techniques with another cultivar to produce a new transgenic, mutant, or edited cultivar. Alternatively, a transgene, mutated gene, or edited gene could be moved into cultivar IPG 110 using traditional breeding techniques, transformation, or gene-editing methods.

Optionally, any of the disclosed methods may further comprise additional steps involving producing peanut seed from the resulting peanut plants and/or planting the peanut seed.

Genetic modifications conferring desirable traits are produced using several methods that are known in the art, including, without limitation, the introduction of polymorphisms, deletions, insertions, mutated genes, converted genes, edited genes, exogenous DNA, and exogenous DNA comprising a native gene or gene element. The genetic modification functions to silence, repress, reduce, or increase the expression of a native gene; or to modify the product produced by a native gene.

The present invention encompasses all plants, or parts thereof, produced by the methods described herein, as well as the seeds produced by these plants. Further, any plants derived from peanut cultivar IPG 110 or produced from a cross using cultivar IPG 110 are provided. The invention also relates to a plant of peanut cultivar IPG 110 comprising a genetic variant, including somaclomal variants, produced through the following methods, without limitation: traditional breeding methods, transformation, mutagenesis, or gene-editing, as well as plants produced in a male-sterile form. Notably, this includes gene-converted plants developed via any of these methods. Thus, the invention relates to plants derived from IPG 110 or variants of IPG 110, but otherwise which have all or essentially all of the physiological and morphological characteristics of IPG 110.

The present invention also encompasses progeny of peanut cultivar IPG 110 comprising a combination of at least two IPG 110 traits selected from those listed in the Tables and Detailed Description of the Invention, wherein the progeny peanut plant is not significantly different from IPG 110 for said traits, as determined at the 5% significance level when grown in the same environment. One of skill in the art knows how to compare a trait between two plant varieties to determine if there is a significant difference between them (Fehr and Walt, Principles of Cultivar Development, pp. 261-286 (1987)). Molecular markers or mean trait values may be used to identify a plant as progeny of IPG 110. Alternatively, progeny may be identified through their filial relationship with peanut cultivar IPG 110 (e.g., as being within a certain number of breeding crosses of peanut cultivar IPG 110). For example, progeny produced by the methods described herein may be within 1, 2, 3, 4, 5, or more breeding crosses of peanut cultivar IPG 110.

Traits of agronomic and/or economic interest include, without limitation: herbicide resistance; insect resistance; resistance to bacterial, fungal, or viral disease; modified fatty acid metabolism; modified carbohydrate metabolism; modified seed yield; yield stability; stress resistance; modified protein percent; modified fancy pod percent; modified pod size, shape, or color; maturity; and male sterility.

The specific gene(s) conferring a trait of interest may be any known in the art or listed herein, including: a polynucleotide conferring resistance to imidazolinone, dicamba, sulfonylurea, glyphosate, glufosinate, triazine, benzonitrile, cyclohexanedione, phenoxy proprionic acid, and L-phosphinothricin; a polynucleotide encoding a Bacillus thuringiensis polypeptide; a polynucleotide encoding phytase, FAD-2, FAD-3, galactinol synthase, or a raffinose synthetic enzyme; or a polynucleotide conferring resistance to rust (Puccinia arachidis), early and late leaf spot (Cercospora arachidicola and Cercosporidium personatum), web blotch (Didymella arachidicola), pepper spot (Leptosphaerulina crassiasca), Tomato Spotted Wilt Tospovirus (TSWV), atmospheric scorch, chemical burn, iron chlorosis, potato leafhopper (Empoasca fabae) seedling disease (Rhizoctonia solani, Pythium spp., Fusarium spp. and others), yellow mold (Aspergillus flavus, Aspergillus parasiticus), root knot nematode (Meloidogyne arenaria) root lesion nematode, southern blight (Sclerotium rolfsii), sclerotinia blight (Sclerotinia minor), Rhizoctonia pod, peg and limb rot (Rhizoctonia solani), pythium pod rot (Pythium myriotylum), botrytis blight (Botrytis cinerea), black mold (Aspergillus niger), blackhull (Thielaviopsis basicola), phymatotrichum root rot (Phymatotrichum omnivorum), and tooth fungus (Phanerochaeta sp).

Any of the seeds, plants, or plant parts provided may be utilized for human food, livestock feed, and as a raw material in industry (see Industrial Uses section below). The present invention also encompasses methods of producing a commodity plant product. Exemplary commodity plant products that can be produced from peanut cultivar IPG 110 include, but are not limited to, edible oil, peanut butter, roasted nuts, salted nuts, livestock feed, flour, soaps, and plastics.

### Tissue Culture

The present invention provides tissue cultures of regenerable cells or protoplasts produced from peanut cultivar IPG 110. As is well known in the art, tissue culture of peanut can be used for the in vitro regeneration of a peanut plant. Thus, such cells and protoplasts may be used to produce plants having the physiological and morphological characteristics of peanut cultivar IPG 110. The peanut plants regenerated by these methods are also encompassed by the present invention.

As used herein, the term “tissue culture” describes a composition comprising isolated cells or a collection of such cells organized into parts of a plant. Exemplary tissues for tissue or cell culture include protoplasts, calli, plant clumps, meristematic cells, and plant cells. Examples of additional plant parts that may be used for tissue or cell culture include embryos, pollen, ovules, hypocotyls, cotyledons, seeds, flowers, glumes, panicles, leaves, stems, shoots, suckers, internodes, buds, roots, root tips, anthers, pedicels, petioles, and pistils. Culture of various peanut cells or tissues and regeneration of plants therefrom is well known in the art.

Methods for culturing plant tissues are known in the art. General descriptions of such methods are provided, for example, by Maki, et al., “Procedures for Introducing Foreign DNA into Plants” in Methods in Plant Molecular Biology & Biotechnology, Glick, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988).

### Breeding Methods

The goal of peanut breeding is to develop new, superior peanut cultivars and hybrids. A superior cultivar is produced when a new combination of desirable traits is formed within a single plant cultivar. Desirable traits may include, but are not limited to, those listed in the Methods section. Single genes may be transferred into the line via the breeding.

The breeding methods used with the present invention may involve a single-seed descent procedure, in which one seed per plant is harvested and used to plant the next generation. Alternatively, the methods may utilize a multiple-seed procedure, in which one or more seeds harvested from each plant in a population is threshed together to form a bulk which is used to plant the next generation.

Use of peanut cultivar IPG 110 in any plant breeding method is encompassed by the present invention. The choice of a breeding or selection method will depend on several factors, including the mode of plant reproduction, the heritability of the trait(s) being improved, and the type of cultivar used commercially (e.g., F₁ hybrid cultivar, pureline cultivar). Popular selection methods include pedigree selection, modified pedigree selection, mass selection, recurrent selection, backcrossing, or a combination thereof.

Pedigree selection is commonly used for the improvement of self-pollinating crops. Two parents are crossed to produce an F₁ population. An F₂ population is produced by selfing one or several F₁'s. Selection of the best individuals may begin in the F₂ population; then, beginning in the F₃ generation, the best individuals in the best families are selected. Replicative testing of families can begin in the F₄ generation to make selection of traits with low heritability more effective. At an advanced stage of inbreeding (e.g., F₆ or F₇), the best lines are tested for potential release as new cultivars.

Mass and recurrent selections can be used to improve populations of either self- or cross-pollinating crops. A genetically variable population of heterozygous individuals is either identified or created by intercrossing several different parents. A genetically variable population may also be created by subjecting a cultivar to mutagenesis. The best plants within the genetically variable population are selected based on individual superiority, outstanding progeny, or excellent combining ability. The selected plants are intercrossed to produce a new population, which often undergoes additional cycles of selection.

Backcrossing is commonly used to transfer genes for highly heritable traits into a desirable homozygous cultivar or variety. The term “backcrossing” refers to the repeated crossing of hybrid progeny back to one of the parental plants, referred to as the recurrent parent. The plant that serves as the source of the transferred trait is called the donor parent. After the initial cross, individuals possessing the transferred trait are selected and repeatedly crossed to the recurrent parent. The resulting plant is expected to have the attributes of the recurrent parent along with the trait transferred from the donor parent.

### Methods of Genetically Modifying Plants

The present invention also encompasses methods of genetically modifying plants of peanut cultivar ‘IPG 110’ to produce peanut varieties comprising essentially all of the physiological and morphological characteristics of ‘IPG 110’ but comprising at least one new trait. Methods of producing a genetically modified peanut plants may rely on any of the recombinant DNA methodology or other methods know to those of skill in the art. For example, plants may be genetically modified by transformation, mutagenesis (including chemical mutagenesis or transposon mutagenesis), genome editing (such as CRISPR/Cas based genome editing or Cre/loxP or other recombinase-based modification) or using RNA interference or gene silencing (via knocking out genes or RNA-based silencing) to genetically modify a peanut plant, or a cell thereof to prepare a genetically modified peanut plant. DNA sequences native to peanut, as well as non-native DNA sequences, can be transformed into peanut and used to alter levels of native or non-native proteins. Various promoters, targeting sequences, enhancing sequences, and other DNA sequences can be inserted into the genome for the purpose of altering the expression of proteins.

The plants may be modified and selected for a wide range of agronomic, physiologic, morphologic or other traits. Traits that may be genetically modified include, but are not limited to, increasing herbicide resistance; insect resistance; or resistance to bacterial, fungal, or viral disease. The plants may also be modified to have modified fatty acid metabolism; modified carbohydrate metabolism; modified seed yield; yield stability; stress resistance; modified protein percent; modified fancy pod percent; modified pod size, shape, or color; or male sterility. These traits may be conferred by increasing or decreasing expression of one or more genes. Insect resistance may include resistance to an insect selected from thrips, southern corn rootworm, burrowing bug, lesser cornstalk borer, leaf hopper, aphid and nematode. Tomato spotted wilt virus and other diseases are transferred to plants by insects. For example, TSWV is transferred most commonly by Tobacco thrips (Frankliniella fusca) and Western flower thrips (Frankliniella occidentalis). The disease resistance may be selected from southern stem rot, late leaf spot, cylindrocladium black rot, sclerotinia blight, early leaf spot, tomato spotted wilt virus and pod rot complex. The modified fatty acid content may be altered concentrations or relative concentrations of oleic acid, linoleic acid and palmitic acid in the peanuts produced by the plants. The modified protein percentage may be altered concentrations or relative concentrations of one or more proteins, including, but not limited to, members of one or more storage protein superfamilies, such as cupin or prolamin superfamilies, and combinations thereof. Examples of storage proteins are Ara h 1, Ara h 2, Ara h 3, Ara h 4, Ara h 5, Ara h 6, Ara h7, Ara h 8, and Ara h 9 (Toomer, O. T. (2017). Nutritional chemistry of the peanut (Arachis hypogaea), Critical Reviews it Food Science and Nutrition. 58(17), 3042-3053. https://doi.org/10.1080/10408398.2017.1339015).

Also encompassed herein are methods of introducing one or more desirable traits of ‘IPG 110’ into another peanut variety. This can be accomplished via conventional breeding methods by crossing the ‘IPG 110’ peanut with anther peanut cultivar that lacks the desirable trait(s) and selecting for progeny plants that contain the desirable trait(s). The selected progeny plants can then be crossed to either parent to produce new progeny and further selected for the desirable trait(s). Further backcrossing can be completed to obtain the progeny containing the desirable trait(s). The desirable trait may be high oleic acid content.

### Transformation Methods

As is noted above, the present invention provides plants and seeds of peanut cultivar IPG 110 in which additional traits have been transferred. While such traits may be selected for using traditional breeding methods, they may also be introduced as transgenes. “Transgenes” include both foreign genes and additional or modified versions of native genes. Plants can be genetically engineered to have a wide variety of traits of agronomic interest. Desirable traits may include without limitation those listed in the Methods section.

Alternatively, transgenic peanut plants in which a gene is silenced (e.g., via knockout, antisense technology, co-suppression; RNA interference, virus-induced gene silencing, targe-RNA-specific ribozymes, hairpin structures, microRNA, and ribozymes) or transgenic peanut plants that express a foreign protein for commercial production may be generated using peanut cultivar IPG 110.

Transgenes are typically introduced in the form of an expression vector. As used herein, an “expression vector” is DNA comprising a gene operatively linked to a regulatory element (e.g., a promoter). The expression vector may contain one or more such gene/regulatory element combinations. The expression vector may also include additional sequences, such as a signal sequence or a tag, that modify the protein produced by the transgene. The vector may be a plasmid and can be used alone or in combination with other plasmids.

Expression vectors include at least one genetic marker operably linked to a regulatory element (e.g., a promoter) that allows transformed cells containing the vector to be recovered by selection. In some embodiments, negative selection, i.e., inhibiting growth of cells that do not contain the selectable marker gene, is utilized. Negative selection markers include, for example, genes that result in detoxification of a chemical agent (e.g., an antibiotic or an herbicide) and genes that result in insensitivity to an inhibitor. Exemplary negative selection genes include neomycin phosphotransferase II (nptII), hygromycin phosphotransferase, gentamycin acetyl transferase, streptomycin phosphotransferase, and aminoglycoside-3′-adenyl transferase. In other embodiments, positive selection, i.e., screening for the product encoded by a reporter gene, is utilized. Exemplary reporter genes include β-glucuronidase, β-galactosidase, luciferase, chloramphenicol acetyltransferase, and Green Fluorescent Protein (GFP).

Transgene expression is typically driven by operably linking the transgene to a promoter within the expression vector. However, other regulatory elements may also be used to drive expression, either alone or in combination with a promoter. As used herein, a “promoter” is a region of DNA upstream of a transcription start site that is involved in recognition and binding of RNA polymerase for transcription initiation. Any class of promoter may be selected to drive the expression of a transgene. For example, the promoter may be “tissue-specific”, “cell type-specific”, “inducible”, or “constitutive”. Those of skill in the art know how to select a suitable promoter based on the particular circumstances and genetic engineering goals.

Methods for producing transgenic plants are well known in the art. General descriptions of plant expression vectors, reporter genes, and transformation protocols can be found in Gruber, et al., “Vectors for Plant Transformation”, in Methods in Plant Molecular Biology & Biotechnology in Glick, et al., (Eds. pp. 89-119, CRC Press, 1993). Methods of introducing expression vectors into plant tissue include direct gene transfer methods, such as microprojectile-mediated delivery, DNA injection, and electroporation, as well as the direct infection, or co-cultivation of plant cells with Agrobacterium tumefaciens, described for example by Horsch et al., Science, 227:1229 (1985). Descriptions of Agrobacterium vector systems and methods for Agrobacterium-mediated gene transfer are provided by Gruber, et al., supra.

In addition, transgenes created in other peanut plants may be transferred in to peanut cultivar IPG 110 using breeding methods (e.g., backcrossing), genetic engineering (e.g., transformation), or via gene editing (e.g., CRISPR-mediated homology-directed repair).

### Mutagenesis Methods

Mutagenesis is another method of introducing new traits into peanut cultivar IPG 110. The goal of artificial mutagenesis is to increase the rate of mutation for a desired characteristic or trait. Desirable traits may include without limitation those listed in the Methods section. Mutation rates can be increased by many different means including temperature, long-term seed storage, tissue culture conditions, radiation (e.g., X-rays, Gamma rays, neutrons, Beta radiation, or ultraviolet radiation), or chemical mutagens (e.g., base analogues such as 5-bromo-uracil or diethyl sulfate), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, and acridines. Once a desired trait is generated through mutagenesis, the trait may then be incorporated into existing germplasm by traditional breeding techniques (e.g., backcrossing). Details of mutation breeding can be found in Fehr, “Principles of Cultivar Development,” Macmillan Publishing Company (1993).

In addition, mutations, including single mutated genes, created in other peanut plants may be transferred into peanut cultivar IPG 110 via genetic engineering (e.g., transformation) or gene editing (e.g., CRISPR-mediated homology-directed repair).

### Gene Editing Methods

In some embodiments, new traits are introduced into peanut cultivar IPG 110 via CRISPR-mediated homology-directed repair. Desirable traits may include without limitation those listed in the Methods section. “Homology directed repair (HDR)” is a naturally occurring nucleic acid repair system that is initiated by the presence of double strand breaks (DSBs) in DNA. In CRISPR-mediated HDR, CRISPR is used to create targeted DSBs (i.e., by targeting a nuclease to cut at specific loci using guide RNAs that are complementary to those loci), which are then repaired using a donor template. The donor template comprises a sequence for insertion flanked by segments of DNA that are homologous to the ends of the DSBs. Thus, in cells that repair the DSBs using the donor template, the genome will be edited to include the sequence for insertion between the sites of the DSBs. Any form of donor template known in the art may be used in the methods of the present invention, including single-stranded oligodeoxynucleotides (ssODNs) and donor plasmids. The nuclease may be naturally existing or engineered. Examples of nucleases include meganucleases, zinc finger nucleases (ZFN), transcription activator-like effector nucleases (TALENs), and the Cas9-guideRNA system (adapted from CRISPR).

In addition, edited genes created in other peanut plants may be transferred into peanut cultivar IPG 110 using breeding methods (e.g., backcrossing), genetic engineering (e.g., transformation), or gene-editing (e.g., CRISPR-mediated homology-directed repair).

### Industrial Uses

Peanuts in the Valencia type market are largely used in peanut butter while peanuts in the Spanish type market are used in certain niche markets where small round peanuts are needed such as confectionery products and red skin peanuts. Peanuts in the runner-type market class are the most commonly used varieties and are found in diverse products such as peanut butter, salted nuts and confectionery products. On the other hand, peanut varieties in the Virginia market class are largely used as salted nuts and in-shell market. Finally, the Peruvian runner market class is grown in certain regions of Mexico.

Peanut is recognized as one of the major oilseed crops and as a rich source of protein. In the United States peanuts are primarily utilized as whole seeds for human foods such as peanut butter, roasted seeds, and confections. In recent years the United States has been the leading exporter of peanuts for human consumption; peanuts rank ninth in area among the row crops and second in dollar value per acre. Peanuts are rich in nutrients, providing over 30 essential nutrients and phytonutrients, and are a good source of niacin, folate, fiber, magnesium, vitamin E, manganese and phosphorus. They are also naturally free of trans-fats and sodium, and contain about 25% protein. Because of these qualities, organizations like the World Health Organization, UNICEF, Project Peanut Butter and Doctors Without Borders have used peanut products to help save malnourished children in developing countries. Thus, improvement of the factors that indicate and/or affect both the food quality of peanuts and the peanut harvest is of considerable importance to the worldwide peanut processing and manufacturing community.

All publications cited in this application are herein incorporated by reference. The foregoing examples of the related art and limitations related therewith are intended to be illustrative and not exclusive. Other limitations of the related art will become apparent to those of skill in the art upon a reading of the specification.

### REFERENCES

Arnarson A., Healthline (healthline.com) (May 7, 2019).
Arya S S, Salve A R, Chauhan S. Peanuts as functional food: a review. J Food Sci Technol. 2016 January; 53 (1): 31-41. doi: 10.1007/s13197-015-2007-9. Epub 2015 Sep. 19. PMID: 26787930; PMCID: PMC4711439.
Fehr, W. R., “Principles of Cultivar Development,” Macmillan Publishing Company, New York, New York (1993).
Fehr, W. R., “Principles of Cultivar Development,” Macmillan Publishing Company, New York, New York (1987).
Gruber, et al., “Vectors for Plant Transformation”, in Methods in Plant Molecular Biology & Biotechnology in Glick, et al., (Eds. pp. 89-119, CRC Press, 1993).
Horsch et al., Science, 227:1229 (1985).
Knauft, D. A. et al., Peanut, Peanut Principles of Cultivar Development, 2:346-384 (Walter R. Fehr ed. 1987).
Maki, et al., “Procedures for Introducing Foreign DNA into Plants” in Methods in Plant Molecular Biology & Biotechnology, Glick, et al., (Eds. pp. 67-88 CRC Press, 1993).
Moore, K. M. et al., J. Heredity 80 (3): 252 (1989).
Norden, A. J., Peanuts, Culture and Uses. Am. Peanut Res. And Educ. Soc., Stillwater, Okla. (C. T. Wilson ed. 1973).
Norden, A. J. in Hybridization of Crop Plants (H. H. Hadley ed. 1980).
Norden, A. J., et al., Breeding of the cultivated peanut in Peanut Science and Technology, (H. E. Pattee ed. 1992).
Norden, A. J. et al., Florida Agr. Res. 3:16-18 (1984).
Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988).
Settaluri, V., C. Kandala, N. Puppala and J. Sundaram, “Peanuts and Their Nutritional Aspects-A Review,” Food and Nutrition Sciences, Vol. 3 No. 12, 2012, pp. 1644-1650. doi: 10.4236/fns.2012.312215.
Singh. B., Singh, U. Peanut as a source of protein for human foods. Plant Food Hum Nutr 41, 165-177 (1991). https://doi.org/10.1007/BF02194085.
Toomer. O. T. Nutritional chemistry of the peanut (Arachis hypogaea). Critical Reviews in Food Science and Nutrition, 58 (17). 3042-3053, 2017). https://doi.org/10.1080/10408398.2017.1339015.
Zhao, T.; Ying, P.; Zhang, Y.; Chen, H.; Yang, X. Research Advances in the High-Value Utilization of Peanut Meal Resources and Its Hydrolysates: A Review. Molecules 2023, 28, 6862. https://doi.org/10.3390/molecules28196862.

### Deposit Information

A deposit of the International Peanut Group proprietary peanut cultivar IPG 110 disclosed above and recited in the claims has been made with the Provasoli-Guillard National Center for Marine Algae and Microbiota (NCMA) (60 Bigelow Drive, East Boothbay, ME 04544). The date of deposit was Dec. 11, 2024. The deposit of 625 seeds was taken from the same deposit maintained by International Peanut Group (1995 County Road 290 Brownfield, TX 79316) since prior to the filing date of this application or the priority application. All restrictions will be irrevocably removed upon granting of a patent, and the deposit is intended to meet all of the requirements of 37 C.F.R. §§ 1.801-1.809. The viability was confirmed as of Dec. 20, 2024. The Accession Number provided by the International Depositary Authority is 202412003. The deposit will be maintained in the depositary for a period of thirty years, or five years after the last request, or for the enforceable life of the patent, whichever is longer, and will be replaced as necessary during that period.

What is claimed is:

1. A seed of Arachis Hypogaea L. peanut cultivar designated ‘IPG 110’, a representative sample of seed of said cultivar having been deposited under Accession No. 202412003 with the NCMA.

2. A peanut plant, or a part thereof, produced by growing the seed of claim 1.

3. A method for producing peanut plants, said method comprising planting a plurality of peanut seeds as recited in claim 1 under conditions favorable for the growth of peanut plants.

4. The method of claim 3, further comprising the step of producing peanut seed from the resulting peanut plants.

5. A peanut seed produced by the method of claim 4.

6. A tissue culture of regenerable cells or protoplasts produced from the peanut plant of claim 2.

7. A peanut plant regenerated from the tissue culture of claim 6, said peanut plant having the morphological and physiological characteristics of ‘IPG 110’.

8. A method for producing an F₁ hybrid peanut plant, said method comprising crossing a first parent peanut plant with a second parent peanut plant, wherein the first parent peanut plant or the second parent peanut plant is the peanut plant of claim 2.

9. The method of claim 8, further comprising the step of producing peanut seed from the resulting peanut plant.

10. A peanut seed produced by the method of claim 9.

11. The method of claim 8, wherein at least one of the first parent peanut plant or second parent peanut plant is transgenic or genome edited.

12. A method of producing a genetically modified peanut plant comprising transforming, mutating, genome editing or using RNA interference or gene silencing to genetically modify the peanut plant of claim 2, or a cell thereof to prepare a genetically modified peanut plant.

13. The method of claim 12, wherein the genetically modified peanut plant is modified to increase herbicide resistance; insect resistance; or resistance to bacterial, fungal, or viral disease; or to have modified fatty acid metabolism; modified carbohydrate metabolism; modified seed yield; yield stability; stress resistance; modified protein percent; modified fancy pod percent; modified pod size, shape, or color; or male sterility.

14. A peanut plant or part thereof, or peanut seed, produced by the method of claim 12.

15. A method of introducing a desired trait into peanut cultivar ‘IPG 110,’ said method comprising the steps of:
a. crossing plants as recited in claim 2 with plants of another peanut line expressing the desired trait, to produce progeny plants;
b. selecting progeny plants that express the desired trait, to produce selected progeny plants;
c. crossing the selected progeny plants with plants from the ‘IPG 110’ parental line to produce new progeny plants;
d. selecting new progeny plants that express both the desired trait and some or all of the physiological and morphological characteristics of peanut cultivar ‘IPG 110,’ to produce new selected progeny plants; and
e. repeating steps (c) and (d) three or more times in succession, to produce selected higher generation backcross progeny plants that express both the desired trait and the physiological and morphological characteristics of peanut cultivar ‘IPG 110,’ when grown in the same environmental conditions.

16. The method of claim 15, additionally comprising the step of planting a plurality of peanut seed produced by selecting higher generation backcross progeny plants under conditions favorable for the growth of peanut plants and optionally comprising the step of producing peanut seed from the resulting peanut plants.

17. The peanut seed resulting from the method of claim 16, wherein, if the resulting peanut seed is grown, then the peanut plants grown from the resulting peanut seed express the desired trait, and wherein the peanut plant otherwise comprises all of the morphological and physiological characteristics of the ‘IPG 110’ peanut plant when grown under the same environmental conditions.

18. A method of producing a commodity plant product, said method comprising obtaining the seed of claim 1, or a part thereof, and producing said commodity plant product therefrom.

19. The method of claim 18, wherein the commodity plant product is selected from the group consisting of edible oil, peanut butter, roasted nuts, salted nuts, raw nuts, confectionary products, flour, livestock feed, biodiesel, fuel, mulch, manufacturing particle board, soaps, fertilizer and plastics.
